(19) United States
(12) Patent Application Publication
(10) Pub. No.: US 2010/0099994 A1
(43) Pub. Date: Apr. 22, 2010

(54) IMPLANTABLE HEART ANALYZING DEVICE, SYSTEM AND METHOD

(76) Inventors: Nils Holmström, Jarfalla (SE); Andreas Blomqvist, Spanga (SE); Berit Larsson, Danderyd (SE); Karin Järverud, Solna (SE); Sven-Erik Hedberg, Kungsangen (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/528,160

(22) PCT Filed: Feb. 28, 2007
(86) PCT No.: PCT/SE07/00193

§ 371 Date: Aug. 21, 2009

(51) Int. Cl. A61B 5/02

(52) U.S. Cl. 600/508

(57) ABSTRACT

An implantable heart analyzing device has a housing and a control circuit located within said housing. The control circuit generates an output signal adapted to actuate an activator, which is able to make a wall of the heart deflect or vibrate. The control circuit also communicates with a sensor, which can be identical with the activator, with which the movement of the heart wall can be sensed. The control circuit executes a procedure that involves the generation of an output signal and sensing a corresponding sensor signal, and to be able to derive information concerning the tension of the heart wall. An implantable heart analyzing includes the aforementioned heart analyzing device, as well as the activator and the sensor. The heart analyzing device and the system implement a method that results in generation of the aforementioned information concerning the tension of the heart wall.

### BACKGROUND OF THE INVENTION

1. Field of the Invention

The present invention relates to an implantable heart analyzing device, for analyzing or monitoring the heart condition.

The invention also concerns an implantable heart analyzing system that includes such an implantable heart analyzing device.

The invention also relates to a method of analyzing or monitoring the heart.

2. Description of the Prior Art

U.S. Pat. No. 5,544,656 describes an ultrasonic sonomicrometer apparatus capable of identifying the myocardial muscle/blood interface and continuously tracking this interface throughout the cardiac cycle using a piezoelectric transducer, which is secured to the epicardium of a ventricle. The apparatus can measure absolute myocardial wall thickness.

WO 2004/066814 A2 describes a cardiac sensor system that includes implanted cardiac sensor assemblies and an external controller which receives information from the implanted sensors. The sensors permit direct measurement of a number of physiologic parameters. The external controller permits calculation of a variety of performance values based on the measured physiological parameters. The system can include a sensor for sensing myocardial elasticity or contractility. This sensor may include a probe which may be mechanically advanced so that it engages myocardial tissue. By applying a known force, and measuring the degree of tissue displacement caused by the force, the elasticity or contractility can be calculated. Conversely, the probe could be moved by a known distance with the amount of required force being tracked in order to calculate elasticity or contractility.

U.S. Pat. No. 6,702,777 describes an apparatus for intracardiac drug administration, including a catheter which is inserted into a chamber of the heart and brought into engagement with a site in the heart wall. The apparatus can include (see column 14, lines 33-59) an ultrasound transducer, which emits a beam of ultrasonic radiation and receives ultrasound waves reflected from the heart wall. The transducer can be used to measure and map the thickness of the heart wall. The transducer may also be used to produce an ultrasound image of the endocardial surface.

WO 2004/096350 A1 describes, in connection with one embodiment in the document (see in particular pages 21-22), a lead with a tip electrode positioned adjacent to the left free ventricular wall. R-wave durations are obtained. For substantially constant ventricular conduction conditions, R-wave duration variations are analyzed to determine ventricular wall thickening of a degree that indicates onset of chronic heart failure.

WO 2006/135294 A1 describes an implantable heart monitoring device comprising a control circuit arranged to generate a vibration signal suitable to actuate a vibrator. The control circuit is also adapted to communicate with at least a first implantable vibration sensor and to receive a detection signal from said vibration sensor. The vibrator is located at a distance from the vibration sensor. The control circuit is arranged to derive information concerning the mechanical properties of the heart, such as the stiffness and/or the thickness of at least a part of the heart.

### SUMMARY OF THE INVENTION

For certain patients it is important to be able to monitor the status of the heart, for example concerning the blood pressure in a certain heart chamber, for example, in the left atrium. An increased blood pressure in the left atrium may be a sign that the heart condition has become worse. An increased blood pressure will also have an effect on the tension of the heart wall surrounding the heart chamber in question. It would therefore be advantageous to be able to detect early symptoms of for example an increased tension in a wall of the heart.

An object of the present invention is to provide an implantable heart analyzing device with which it is possible to derive information concerning the tension of a heart wall. A further object is to provide such a device that is able to monitor changes in the tension of the heart wall. Still an object of the invention is to provide such a device with a relatively simple construction.

The above objects are achieved by an implantable heart analyzing device having a housing and a control circuit located within the housing.

The control circuit being adapted to generate an output signal adapted to actuate an activator, which is adapted to be positioned in contact with an inner or an outer wall of the heart, such that the output signal, when the device is connected to the activator positioned in contact with the wall of the heart, is able to make the wall of the heart deflect or vibrate.

The control circuit also being adapted to communicate with a sensor, which is either identical with the activator or which constitutes a separate sensor, that is adapted to be positioned in contact with the same wall of the heart as the activator, wherein the control circuit is adapted to from the sensor receive a sensor signal, which, when the sensor is positioned in contact with the wall of the heart, represents the movement of said heart wall.

The control circuit is arranged to be able to carry out a procedure that involves the generation of at least one said output signal and sensing, during a certain time interval, a corresponding sensor signal, and wherein the control circuit is set up to be able to derive, based on the shape of the sensor signal sensed in response to the generated output signal, information concerning the tension of the heart wall.

With the use of the sensor signal, it is thus possible to detect the movement of the heart wall in response to a deflection or vibration generated with the help of the activator. The sensor signal therefore contains information of how the heart wall in question moves. The movement of the heart wall depends, inter alia, on the tension of the heart wall. With the help of a device according to the present invention, it is therefore possible to obtain information about the tension of the heart wall in question and thereby also information about the mechanical properties of the heart wall.

An early indication of a dysfunction of the heart may be an increased blood pressure in a certain heart chamber, for example in the left atrium. This increased blood pressure will also have an effect on the tension in the heart wall. With the present invention it is possible, for example, to detect early a sign of an increased blood pressure in a certain heart chamber. This problem can therefore be treated at an early stage if the detection has been done early with the help of the present invention. An early detection, and a treatment, of for example increased blood pressure in the left atrium may make it possible to avoid the development of a severe congestive heart failure in the patient.

It should be noted that from some of the prior art documents noted above it is known to transmit an ultrasound wave and to analyze, for example, the time it takes for this wave to reach the detector. The present invention is not at all concerned with this kind of analysis. Instead, the present invention concerns the sensing of the actual behaviour of a heart wall (how the heart wall moves or vibrates) in response to a triggered deflection or vibration of the heart wall. This behavior is detected by sensing the mentioned sensor signal.

With an inner wall of the heart is in this document meant a wall in the interior of the heart, for example the septum between the atria or the septum between the ventricles.

With an outer wall of the heart is in this document meant a wall of the heart that is not located in the interior of the heart. In order to cause an outer wall of the heart to deflect or vibrate, the activator can for example with advantage be positioned inside the pericardium on the epicardium, or possibly outside the pericardium. The activator could, according to a less preferred embodiment, also be positioned in the heart (in an atrium or a ventricle) in contact with an outer wall.

The “shape” of the sensor signal encompasses any information related to the tension of the heart wall, which is contained in the sensor signal. This information can be, for example, the attenuation of the sensor signal.

According to an embodiment of the implantable heart analyzing device according to the invention, the device comprises at least one memory connected to the control circuit, wherein the control circuit is arranged to derive the information concerning the tension by comparing the shape of the sensor signal with a shape that has previously been stored in the memory. In the memory it is thus possible to store a type of “template” that represents a typical sensor signal. A certain deviation from this stored template may then be an indication of, for example, the fact that the tension in the heart wall in question has increased.

According to a further embodiment of the implantable heart analyzing device, the control circuit is arranged to be able to operate in time cycles corresponding to heart cycles. It is well known for example in connection with heart stimulating devices that such devices operate in time cycles corresponding to heart cycles.

According to a further embodiment, the control circuit is arranged such that said procedure is to be carried out during a portion of said time cycle that corresponds to a late part of the diastolic portion of the heart cycle. The diastolic portion can either be an atrial diastole or a ventricular diastole. It has thus appeared to be advantageous to measure the tension of the hear wall during such portions of the heart cycle. However, the device can also be arranged to carry out the procedure in question during other portions of the heart cycle.

If the activator/sensor is attached to an atrial wall, the procedure can for example be carried out around time of the P-wave (for example later than 100 ms before the expected occurrence of the P-wave, but before 50 ms after the P-wave). If the activator/sensor is attached to a ventricular wall, the procedure can for example be carried out around the time of the QRS-complex (for example later than 50 ms before the expected occurrence of the QRS-complex, but before 150 ms after the QRS-complex).

According to a further embodiment, the control circuit is arranged to carry out said procedure at a plurality of occasions, and to store a result from said procedure in said memory, such that information can be derived of how said tension of the heart wall has changed between these occasions. It is thereby possible to monitor how the tension of the heart wall in question has changed over time.

According to a further embodiment, the control circuit is arranged such that said procedure is carried out at the same portion of said time cycle at the different occasions. In order to monitor how the tension of the heart wall changes over time, it is advantageous to carry out the procedure during the same portion of the heart cycle.

According to a further embodiment, the control circuit is arranged such that said procedure is carried out at a plurality of occasions within the same time cycle. By carrying out the procedure at different occasions within the same time cycle, it is possible to detect how the tension of the heart wall has changed during the same time cycle. It is, for example, possible to first carry out the procedure during the time of the QRS-complex and then at a second occasion after the contraction of the heart.

According to a further embodiment, the control circuit is arranged such that a relationship between at least two such sensor signals within the same time cycle is stored in a memory. It is hereby possible to obtain information of for example the ratio between the tensions at two different occasions within the same time cycle. Also such a relationship may provide information about the condition of the heart. For example, in a patient suffering from congestive heart failure it appears that the tension in the heart wall may remain to a certain extent also during the diastolic portion of the heart cycle.

According to a further embodiment, the control circuit is arranged such that said relationship is formed during different time cycles, such that information can be derived of how said relationship has changed between these different time cycles. It is hereby possible to see how the mentioned relationship changes over time. A certain change of the relationship (for example the mentioned ratio) may indicate that the heart condition has become worse.

According to a further embodiment, the control circuit is arranged such that the output signal is a short pulse or voltage step. It has appeared to be advantageous to actuate the activator with such a short pulse or voltage step.

According to a further embodiment, said activator is a piezoelectric device. With advantage, a piezoelectric device can be used as the activator.

According to a further embodiment, the control circuit is arranged such that said time interval begins within 30 ms after the generation of said output signal. It is advantageous to start the sensing very soon after the generation of the output signal, while the heart still moves or vibrates substantially in response to the activation caused by the activator.

According to a further embodiment, the control circuit is arranged such that said time interval is less than 200 ms long. It has also appeared to be advantageous to only carry out the detection procedure during a short time interval.

The mentioned time interval, during which the sensor signal is sensed is with advantage at least 20 ms long, preferably more than 30 ms long. The time interval is however, with advantage less than 150 ms long, preferably less than 100 ms long. The time interval can for example be 80 ms long. Preferably, the time interval begins directly after that the output signal has been delivered.

According to a further embodiment, the control circuit is arranged such that the attenuation of the sensor signal during said time interval is used as a measure of the tension of the heart wall. It has appeared to be advantageous to use the attenuation of the sensor signal as an indication of the tension of the heart wall. However, also other features of the sensor signal may be used as an indication of the tension of the heart wall.

Another aspect of the present invention concerns an implantable heart analyzing system. According to the invention, this system comprises an implantable heart analyzing device according to any one of the preceding embodiments together with said activator and said sensor, which is either identical with said activator or which constitutes a separate sensor, wherein the control circuit is set up to communicate with said activator and sensor. Such a system according to the invention has advantages corresponding to those described above in connection with the device.

Another aspect of the present invention concerns a method of analyzing the tension of a wall of a heart in a human or animal being. The method includes the following steps: position an activator in contact with an inner or an outer wall of the heart, position a sensor in contact with the same wall of the heart as the activator, wherein the sensor is either identical with the activator or constitutes a separate sensor, carry out a procedure which involves the generation of at least one output signal that actuates the activator, such that the activator makes said wall of the heart deflect or vibrate, and sensing, during a certain time interval, a corresponding sensor signal from the sensor, which sensor signal represents the movement of the heart wall, and deriving, based on the shape of the sensor signal sensed in response to the generated output signal, information concerning the tension of the heart wall.

The method according to the invention has similar advantages to those described above in connection with the device according to the invention.

### BRIEF DESCRIPTION OF THE DRAWINGS

FIG. 1 schematically illustrates an embodiment of an implantable heart analyzing device according to the invention, connected to leads with sensing and pacing electrodes, and an activator and a sensor positioned in or at the heart of a patient.

FIG. 2 schematically illustrates an embodiment of a control circuit for use in the device according to the invention.

FIG. 3 schematically illustrates a signal for actuating the activator, as well as a subsequent sensor signal.

FIG. 4 schematically illustrates the same type of signal shown in FIG. 3, but with a different sensor signal obtained at a different time from the sensor signal shown in FIG. 3.

FIG. 5 schematically illustrates a further manner for actuating the activator in accordance with the present invention.

FIG. 6 schematically illustrates an embodiment of a method according to the present invention.

FIG. 7 schematically illustrates a further embodiment of the method according to the invention.

### DESCRIPTION OF THE PREFERRED EMBODIMENTS

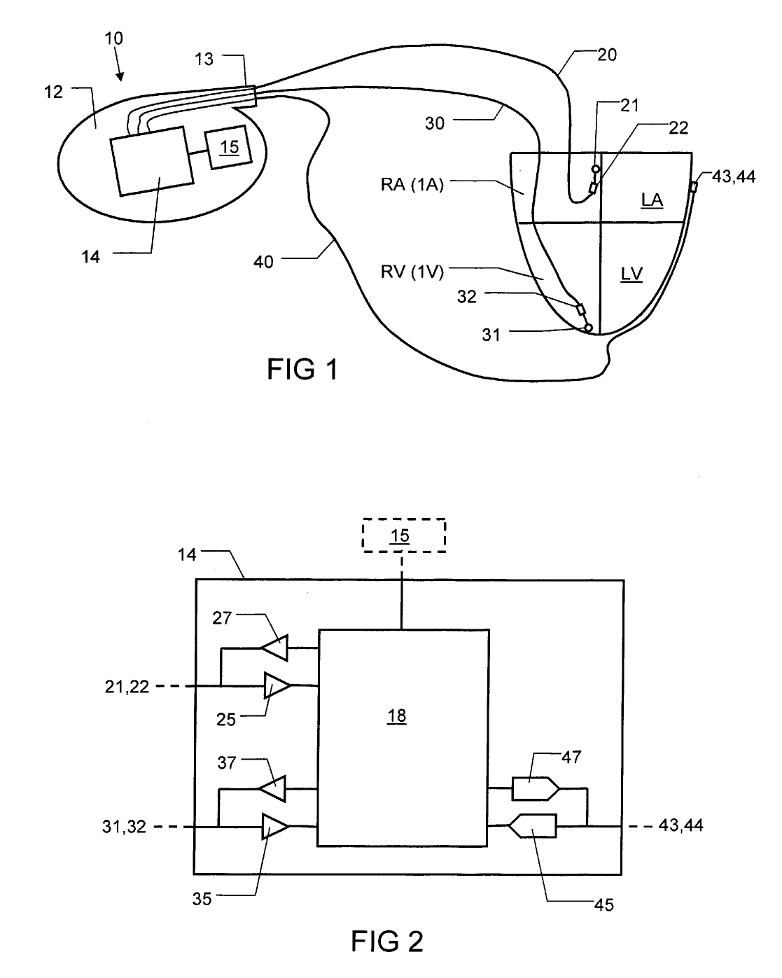

FIG. 1 shows schematically an implantable heart analyzing device 10 according to the invention. According to the shown embodiment, the device 10 also functions as a heart stimulating device. The device 10 has a housing 12. The device 10 has a connector portion 13. Via the connector portion 13, the device 10 can be connected to different leads. In FIG. 1 the device 10 is connected to three leads 20, 30 and 40.

FIG. 1 also schematically illustrates a heart with a right atrium RA, a left atrium LA, a right ventricle RV and a left ventricle LV.

The lead 20 includes a pacing and sensing electrode 21, 22.

Similarly to the lead 20, the lead 30 includes a pacing and sensing electrode 31, 32.

The lead 40 is connected to an activator 43 in the form of a piezoelectric device. According to this embodiment, the activator 43 also acts as a sensor 44 for sensing the movement of a heart wall. However, according to an alternative embodiment, the sensor 44 can be separate from the activator 43. If the sensor 44 is separate from the activator 43, the sensor 44 should preferably still be positioned close to the activator 43 at the same heart wall as the activator 43. In the shown embodiment, the activator/sensor 43, 44 is positioned on the heart wall outside the left atrium LA. The activator/sensor 43, 44 may for example be introduced into the pericardium and be positioned in the pericardium on the epicardium at the left atrium. However, it is also possible that the activator/sensor 43, 44 is introduced into the heart and positioned for example at or in the septum between the left and right atria.

In the shown example, the electrodes 21, 22, 31, 32 are bipolar electrodes with a tip portion 21, 31 and a ring portion 22, 32. However, it is also possible to use unipolar electrodes. This is known to those skilled in the art.

The device 10 comprises a control circuit 14, which will be described further below. The device 10 also comprises at least one memory 15 connected to the control circuit 14.

The device 10 together with the leads 20, 30, 40 and the pacing/sensing electrodes 21, 22, 31, 32 and the activator/sensor 43, 44 constitute a heart stimulating system according to the invention. This system can be implanted in a patient.

It can be noted that FIG. 1 shows a device 10 which can electrically pace and sense both an atrium and a ventricle. However, also other alternatives are possible. The device 10 can of course also constitute an analyzing device 10, which is not designed to pace and sense the heart.

In FIG. 1, the electrode 21, 22 constitutes a first atrial sensing and/or pacing electrode 21, 22 which is positioned in a first atrium 1A of the heart, according to this embodiment the right atrium RA, in order to enable sensing and/or pacing of this atrium RA.

The electrode 31, 32 constitutes a first ventricular sensing and pacing electrode 31, 32, which is positioned in a first ventricle 1V of the heart, in this embodiment the right ventricle RV. The first ventricular sensing and pacing electrode 31, 32 is adapted to enable sensing and pacing of this first ventricle 1V.

It is also possible that the device 10 is connected to further leads and/or further sensing/pacing electrodes, for example electrodes positioned in order to sense and/or pace the left ventricle LV and/or the left atrium LA and electrodes designed to enable defibrillation.

FIG. 2 shows schematically the control circuit 14 in some more detail. The control circuit 14 includes a control portion 18 connected to a memory 15.

The control circuit 14 includes a first atrial sensing and/or pacing circuit 25, 27. In this embodiment, this circuit 25, 27 includes a sensing circuit 25 and a pacing circuit 27. The first atrial sensing and/or pacing circuit 25, 27 communicates with the first atrial sensing and/or pacing electrode 21, 22 via the lead 20. The first atrial sensing and/or pacing circuit 25, 27 is thus adapted to sense and/or pace a first atrium 1A, in this case the right atrium RA.

The control circuit 14 also includes a first ventricular sensing circuit 35 and a first ventricular pacing circuit 37. These circuits 35, 37 communicate with the first ventricular sensing and pacing electrode 31, 32 via the lead 30. The circuits 35, 37 are thus adapted to sense and pace a first ventricle 1V, in this case the right ventricle RV.

Although not described in any detail here, the device 10 can be arranged to include several other operational features that are known in connection with heart stimulation/analyzing devices. Such normal features include, for example that the control portion 18 is set up to operate with blanking and refractory periods; the ability to deliver back-up pulses if a heart chamber is not captured when a pacing pulse has been delivered; the ability to perform capture threshold searches; the ability to carry out defibrillation; an activity sensor for sensing the activity of the patient in whom the device has been implanted; the ability to sense impedance values; the ability to sense chemical substances in the blood; the ability to communicate with the use of so-called telemetry, etc.

The control circuit 14 is arranged to be able to operate in time cycles corresponding to heart cycles. Such an operation is normal for an implantable heart stimulating device. The time cycles are determined by preset timer intervals which also may depend on detected signals.

The control circuit 14 also comprises a circuit 47 adapted to generate an output signal in order to actuate the activator 43. Furthermore, the control circuit 14 comprises a circuit 45 adapted to receive a sensor signal from the sensor 44 and to thereby detect the movement of the heart wall on which the sensor 44 is positioned.

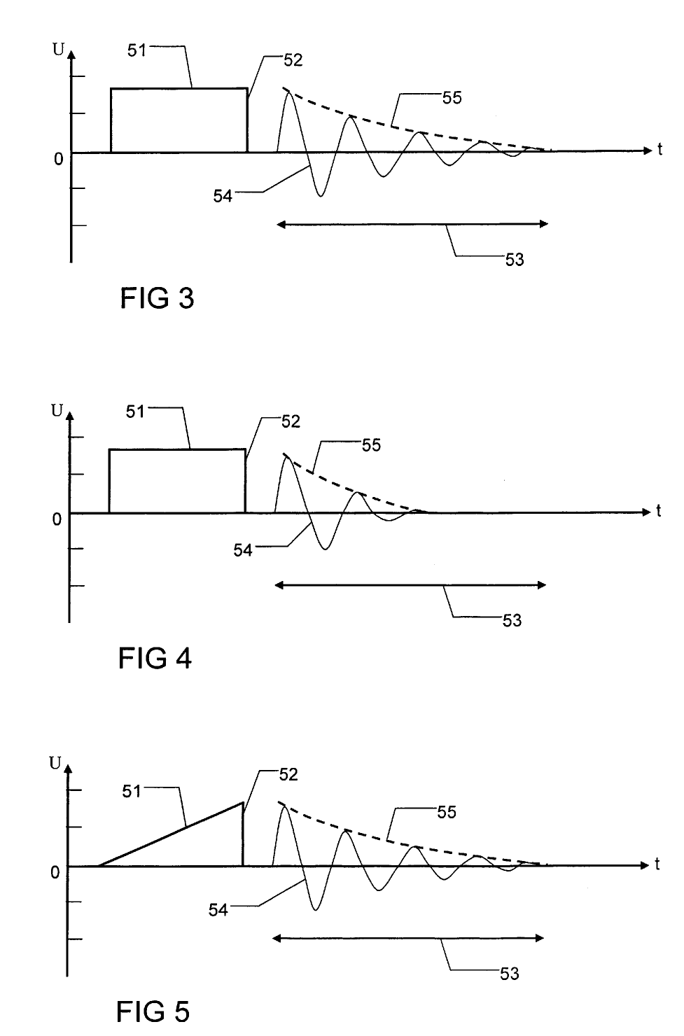

FIG. 3 shows voltage U as a function of time t and illustrates schematically how an output signal to the activator 43 can be generated. The activator 43 can thus be a piezoelectric crystal or other piezoelectric device. The control circuit 14 is arranged to apply a voltage 51 over the activator 43. When the circuit to the activator 43 is short-circuited (shown at 52) or reversed the thickness (or the deflection) of the activator 43 changes rapidly. The voltage drop 52 thus constitutes an output signal to the activator 43 which causes the activator to make the wall of the heart deflect or vibrate.

During a certain time interval (sensing window) 53, the control circuit 14 senses a sensor signal 54 which represents the movement of the heart wall. The sensor signal 54 is received from the sensor 44, which may be the same device as the activator 43. The sensing window 53 may for example start 10 ms after the generation of the output signal 52. The sensing window 53 may for example be 80 ms long. The shape of the sensor signal 54 contains information concerning the tension of the heart wall. The attenuation of the sensor signal 54 is represented by 55. The attenuation 55 of the sensor signal is an indication of the tension of the heart wall.

In FIG. 3 it is shown that the voltage of the sensor signal 54 has both positive and negative values. This is an indication of the fact that the heart wall vibrates. Also the frequency of this vibration is related to the tension of the heart wall. It should however be noted that it is not always the case that the heart wall will vibrate in response to an actuation caused by the activator 53. Instead it is possible that the heart wall regains its previous shape without vibration. However, even in this case, the time it takes for the heart wall to regain its shape is an indication of the tension. Information concerning the tension of the heart wall can also be obtained by comparing the shape of the sensor signal 54 with the shape of a template that has previously been stored in the memory 15.

The control circuit 14 is arranged or programmed to carry out the procedure illustrated in FIG. 3 at different occasions. This procedure can, for example, when an atrial heart wall is sensed, be carried out around the time of the P-wave, or at a late part of the diastolic portion of the heart cycle.

The control circuit 14 can be arranged to carry out the procedure at a plurality of occasions, and to, at each occasion, store the result from the procedure in the memory 15. In this way, information can be derived of how the tension of the heart wall has changed between these occasions.

FIG. 4 illustrates schematically the procedure corresponding to FIG. 3 performed at a later occasion. As can be seen in FIG. 4, the sensor signal 54 is more attenuated in FIG. 4 than in FIG. 3. This is an indication of the fact that the tension of the heart wall in question has changed.

Preferably, the control circuit 14 is arranged such that the procedure is carried out at the same portion of the time cycle at the different occasions.

It is also possible that the control circuit 14 is arranged such that the procedure is carried out at a plurality of occasions within the same time cycle. For example, the procedure can be carried out at two different occasions within the same heart cycle. The control circuit 14 can thereby be arranged such that a relationship between the two sensor signals from these two occasions is stored in the memory 15. The relationship may for example be the ratio between the attenuations 55.

The control circuit 14 can be arranged such that this relationship is formed during different time cycles. In this way it is possible to derive information of how the relationship has changed between the different time cycles.

FIG. 5 illustrates schematically a slightly different manner of producing the output signal 52 than the manner shown in FIG. 3. According to FIG. 5, the voltage over the piezoelectric activator 43 is gradually increased. Then the circuit is short-circuited in order to provide the output signal 52.

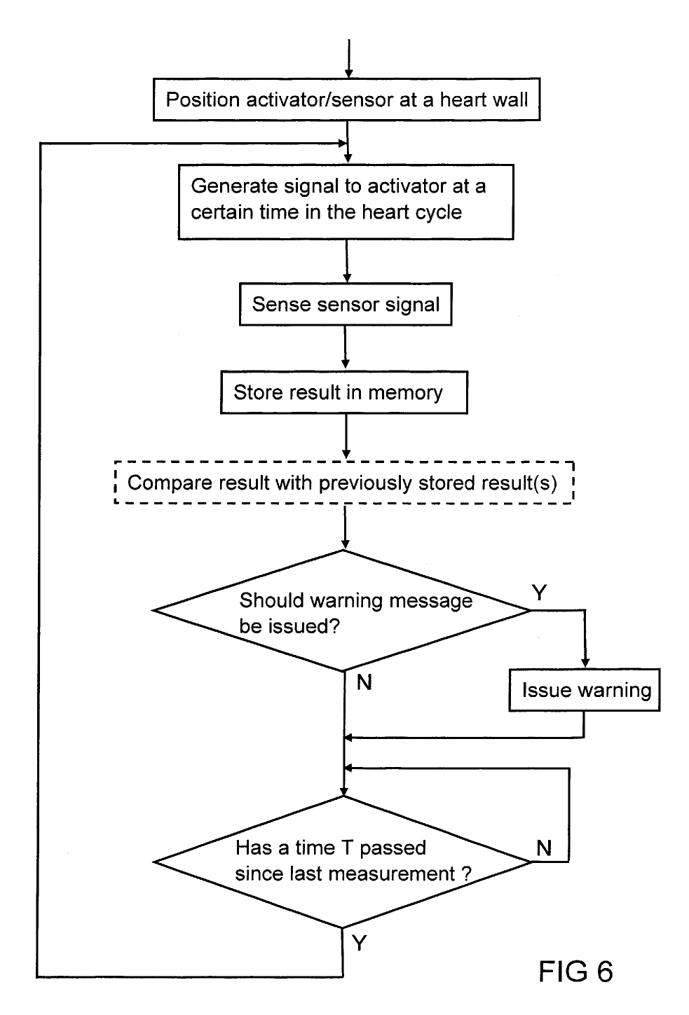

A manner of carrying out the method according to the invention will now be described with reference to FIG. 6.

An activator 43 is positioned in contact with an inner or an outer wall of the heart. The activator 43 can, for example, be positioned in the pericardium of the heart, for example in contact with the epicardium outside the left atrium LA. Alternatively, the activator 43 can be positioned in the heart, for example in the septum between the atria.

A sensor 44 is positioned in contact with the same wall of the heart as the activator 43. The sensor 44 can be identical with the activator 43. The activator 43 and the sensor 44 may be piezoelectric devices.

A procedure is carried out in order to obtain information concerning the tension of the heart wall. This procedure can, for example, be carried out during a late part of the diastolic portion of the heart cycle. If an atrial heart wall is to be sensed, the procedure can, for example, be performed about the time of the P-wave.

This procedure involves the generation of an output signal that actuates the activator 43. The output signal can be a short voltage step 52. The activator 43 will cause the wall of the heart to deflect or vibrate, preferably by producing a short impulse on the heart wall. A corresponding sensor signal 54 from the sensor 44 is then sensed during a certain time interval 53. The time interval 53 can, for example, begin 10 ms after the generation of the output signal 52. The time interval 53 can, for example, be 80 ms long. The sensor signal 54 represents the movement of the heart wall. Based on the shape of the sensor signal 54 sensed in response to the generated output signal 52, information concerning the tension of the heart wall can be derived. This can be done, for example, by comparing the shape of the sensor signal 54 with a shape that has previously been stored in a memory 15. For example the attenuation of the sensor signal 54 during the time interval 53 can be used as a measure of the tension of the heart wall. The result of the procedure is stored in the memory 15.

The method may also include the generation of a warning message, for example if the tension of the heart wall is higher than a predetermined value or if the tension has changed more than a predetermined amount. The warning message can for example be that a message is stored in the memory 15, such that this message can later be retrieved by the physician. The warning message may also be provided to a health care provider via Radio Frequency Telemetry to e.g. a home monitoring unit that relays the warning information to the health care provider. Alternatively, the warning, for example in the form of a vibration of the hosing 12, could alert the patient.

The method so far described can be carried out again if a certain time has passed. The procedure can thus be carried out at a plurality of occasions, for example once a day. Preferably, the procedure is carried out at the same portion of the heart cycle at the different occasions. At each occasion, the result from said procedure is stored in a memory 15. Hereby, it is possible to derive information of how the tension of the heart wall has changed between these occasions. It should be noted that at each “occasion” the procedure can in fact be carried out a number of times, in order to obtain a statistically more correct value of the tension of the heart wall.

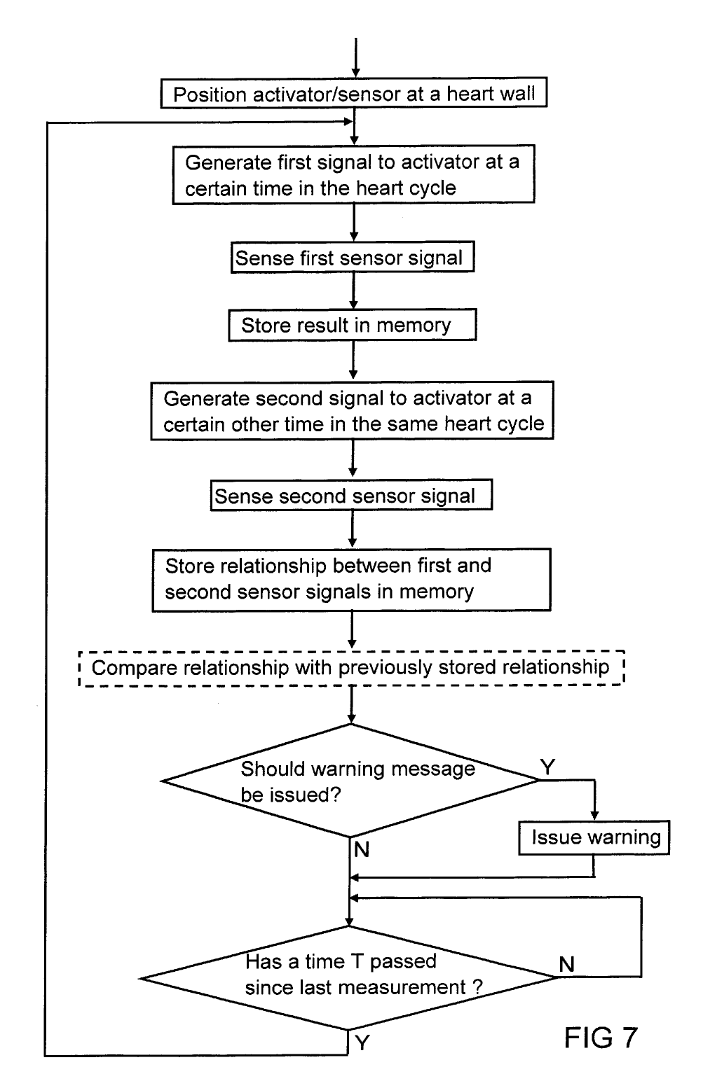

FIG. 7 illustrates another manner of carrying out the method according to the invention. This manner is basically the same as the one illustrated in FIG. 6. However, the method of FIG. 7 differs from the method of FIG. 6 in that the mentioned procedure is carried out twice within the same heart cycle, for example once about the time of the P-wave and once after the contraction of the heart. It is of course within the scope of the invention that the procedure is carried out more than two times during the same heart cycle.

The result from each procedure is stored in the memory 15. According to one possible manner of carrying out the method, a relationship between at least two such sensor signals 54 within the same heart cycle is stored in the memory 15. This relationship can for example be the ratio between the attenuations at the two different procedures.

Similarly to the method of FIG. 6, a warning message can be issued, for example if the mentioned relationship fulfils a predetermined criteria.

Also analogously to the method illustrated in FIG. 6, the whole method described so far can be carried out at regular intervals, for example once a day. The relationship can thus be formed during different heart cycles, such that information can be derived of how said relationship has changed between these different heart cycles. The warning message can thus for example be issued if the mentioned relationship has changed more than a predetermined amount between the different times when the method is carried out.

Although modifications and changes may be suggested by those skilled in the art, it is the intention of the inventors to embody within the patent warranted heron all changes and modifications as reasonably and properly come within the scope of their contribution to the art.

1.-30. (canceled)

31. An implantable heart analyzing device comprising:
a housing configured for in vivo implantation in a patient;
a control circuit contained within said housing;
said control circuit comprising output circuitry configured to electrically communicate with an activator positioned in contact with a heart wall, selected from the group consisting of an inner wall and an outer wall, of the heart of the subject, said output circuitry being configured to generate an output signal that causes said activator to deflect or vibrate said heart wall;
said control circuit comprising input circuitry configured to electrically communicate with a sensor that supplies a sensor signal to said input circuitry representing movement of said heart wall; and
said control circuit being configured to implement a session comprising generating at least one output signal with said output circuitry and supplying said output signal from said output circuitry to said activator, and detecting said sensor signal representing movement of the heart wall caused by said activator within a predetermined time interval, said sensor signal having a sensor signal morphology, and to automatically evaluate said sensor signal morphology to identify information representing tension of said heart wall.

32. An implantable heart analyzing device as claimed in claim 31 comprising a memory in said implantable device connected to said control circuit, said memory having information electronically stored therein representing a stored sensor signal morphology, and wherein said control circuit determines said information representing tension of said heart wall by comparing the sensor signal morphology represented by the detected sensor signal with the stored sensor signal morphology in said memory.

33. An implantable heart analyzing device as claimed in claim 31 wherein said control circuit is configured to implement said session in time cycles corresponding to heart cycles of the heart of the subject.

34. An implantable heart analyzing device as claimed in claim 33 wherein said control circuit is configured to implement said session during a portion of said time cycle corresponding to a late portion of the diastolic phase of the heart cycle of the heart of the subject.

35. An implantable heart analyzing device as claimed in claim 31 comprising a memory connected to said control circuit in said housing, and wherein said control circuit is configured to implement said session a plurality of times respectively in different heart cycles of the heart of the subject, and to obtain said information describing tension of the heart wall in each session as a session result, and to store the respective session results in said memory, and to evaluate the respective session results in said memory to determine a change in said tension of the heart wall between different sessions.

36. An implantable heart analyzing device as claimed in claim 35 wherein said control circuit is configured to implement said session in a same part of each of said different heart cycles.

37. An implantable heart analyzing device as claimed in claim 35 wherein said control circuit is configured to implement said session multiple times within a single heart cycle of the heart of the subject.

38. An implantable heart analyzing device as claimed in claim 37 wherein said control circuit is configured to identify a relationship between at least two of the sensor signals for respective sessions within said single heart cycle, and to store said relationship in said memory.

39. An implantable heart analyzing device as claimed in claim 38 wherein said control circuit is configured to implement said session multiple times in each of a plurality of different heart cycles of the heart of the subject, and to determine said relationship of at least two of said sensor signals respectively obtained in different ones of said plurality of heart cycles, and to determine a change in said relationship between said different ones of said heart cycles.

40. An implantable heart analyzing device as claimed in claim 31 wherein said output circuitry is configured to generate said output signal in a form selected from the group consisting of pulses and voltage steps.

41. An implantable heart analyzing device as claimed in claim 31 wherein said activator is a piezoelectric device.

42. An implantable heart analyzing device as claimed in claim 31 wherein said control circuit is configured to begin said time interval within 30 ms after generating said output signal with said output circuitry.

43. An implantable heart analyzing device as claimed in claim 31 wherein said control circuit is configured to set said time interval as being less than 200 ms in duration.

44. An implantable heart analyzing device as claimed in claim 31 wherein said control circuit is configured to evaluate the morphology of the sensor signal by identifying an attenuation of the sensor signal within said time interval as a measure of said tension of the heart wall.

45. An implantable heart analyzing system comprising:
an activator configured for in vivo placement on a heart wall, selected from the group consisting of an inner heart wall and an outer heart wall, of the heart of a subject, said activator being operable to cause said heart wall to deflect or vibrate;
a sensor configured for in vivo placement at said heart wall to detect movement of said heart wall;
a housing configured for in vivo implantation in the subject;
a control circuit contained in said housing, said control circuit comprising output circuitry electrically connected to said activator and input circuitry electrically connected to said sensor;
said output circuitry being configured to generate an output signal that causes said activator to deflect or vibrate said heart wall;
said input circuitry being configured to detect, within a predetermined time interval, a sensor signal from said sensor representing movement of said heart wall in response to operation of said activator, said sensor signal having a sensor signal morphology; and
said control circuit being configured to implement a session, comprising generating said output signal and detecting said sensor signal in said time interval, and to evaluate said sensor signal morphology to derive information representing tension of said heart wall.

46. An implantable heart analyzing system as claimed in claim 45 wherein said activator is the same component as said sensor.

47. A method for monitoring a heart comprising the steps of:
implanting an activator in vivo in contact with a heart wall, selected from the group consisting of an inner wall and an outer wall, of the heart of the subject; said
placing said activator in communication with a control circuit and, from output circuitry of said control circuit, generating an output signal that causes said activator to deflect or vibrate said heart wall;
implanting a sensor in vivo at a location to sense movement of said heart wall and placing said sensor in communication with said control circuit and supplying a sensor signal to input circuitry of said control circuit representing said movement of said heart wall; and
in said control circuit, implementing a session comprising generating at least one output signal with said output circuitry and supplying said output signal from said output circuitry to said activator, and detecting said sensor signal representing movement of the heart wall caused by said activator within a predetermined time interval, said sensor signal having a sensor signal morphology, and automatically evaluating said sensor signal morphology to identify information representing tension of said heart wall.

48. A method as claimed in claim 47 comprising, in a memory in communication with said control circuit, storing information representing a stored sensor signal morphology and, in said control circuit, determining said information representing tension of said heart wall by comparing the sensor signal morphology represented by the detected sensor signal with the stored sensor signal morphology in said memory.

49. A method as claimed in claim 47 comprising, in said control circuit, implementing said session in time cycles corresponding to heart cycles of the heart of the subject.

50. A method as claimed in claim 49 comprising, in said control circuit, implementing said session during a portion of said time cycle corresponding to a late portion of the diastolic phase of the heart cycle of the heart of the subject.

51. A method as claimed in claim 47 comprising placing said control circuit in communication with a memory and, in said control circuit, implementing said session a plurality of times respectively in different heart cycles of the heart of the subject, and obtaining said information describing tension of the heart wall in each session as a session result, and storing the respective session results in said memory, and evaluating the respective session results in said memory to determine a change in said tension of the heart wall between different sessions.

52. A method as claimed in claim 50 comprising, in said control circuit, implementing said session in a same part of each of said different heart cycles.

53. A method as claimed in claim 50 comprising, in said control circuit, implementing said session multiple times within a single heart cycle of the heart of the subject.

54. A method as claimed in claim 53 comprising, in said control circuit, identifying a relationship between at least two of the sensor signals for respective sessions within said single heart cycle, and to storing said relationship in said memory.

55. A method as claimed in claim 54 comprising, in said control circuit, implementing said session multiple times in each of a plurality of different heart cycles of the heart of the subject, and determining said relationship of at least two of said sensor signals respectively obtained in different ones of said plurality of heart cycles, and determining a change in said relationship between said different ones of said heart cycles.

56. A method as claimed in claim 47 comprising, from said output circuitry, generating said output signal in a form selected from the group consisting of pulses and voltage steps.

57. A method as claimed in claim 47 comprising employing a piezoelectric device as said activator.

58. A method as claimed in claim 47 comprising, in said control circuit, beginning said time interval within 30 ms after generating said output signal with said output circuitry.

59. A method as claimed in claim 47 comprising, in said control circuit, setting said time interval as being less than 200 ms in duration.

60. A method as claimed in claim 47 comprising, in said control circuit, evaluating the morphology of the sensor signal by identifying an attenuation of the sensor signal within said time interval as a measure of said tension of the heart wall.

61. A method as claimed in claim 47 comprising employing the same component as said analyzer and said sensor.

62. A method as claimed in claim 47 comprising placing said activator in the pericardium of the heart of the subject.

63. A method as claimed in claim 47 comprising placing said activator in the septum between chambers in the heart of the subject.

64. A computer-readable medium encoded with programming instructions that operate a control circuit of an implantable heart analyzing system comprising an activator configured for in vivo placement on a heart wall, selected from the group consisting of an inner heart wall and an outer heart wall, of the heart of a subject, said activator being operable to cause said heart wall to deflect or vibrate, a sensor configured for in vivo placement at said heart wall to detect movement of said heart wall, and said control circuit comprising output circuitry electrically connected to said activator and input circuitry electrically connected to said sensor, said programming instructions causing said control circuit to:
from said output circuitry, generate an output signal that causes said activator to deflect or vibrate said heart wall;
with said input circuitry, detect, within a predetermined time interval, a sensor signal from said sensor representing movement of said heart wall in response to operation of said activator, said sensor signal having a sensor signal morphology; and
implement a session, comprising generating said output signal and detecting said sensor signal in said time interval, and evaluate said sensor signal morphology to derive information representing tension of said heart wall.
